(19) United States
(12) Patent Application Publication
(10) Pub. No.: US 2026/0345444 A1
(43) Pub. Date: Nov. 5, 2026

(54) FULLY DISSOLVABLE ANTICARIOUS REMINERALIZING DENTAL PATCH FOR CHILDREN AND PREPARATION METHOD THEREOF

(71) Applicant: Jiangxi Dentalbright Technology Co., Ltd, Jiujiang (CN)

(72) Inventors: Junyuan Qin, Nanchang (CN); Wei Min, Nanchang (CN); Yongbin Yuan, Ji'an (CN)

(21) Appl. No.: 19/707,976

(22) Filed: Jun. 15, 2026

(30) Foreign Application Priority Data
May 20, 2026 (CN) 202610701376.8

(51) Int. Cl. A61K 6/20; A61K 6/15; A61K 6/65; A61K 6/69; A61K 6/70

(52) U.S. Cl. CPC A61K 6/20; A61K 6/15; A61K 6/65; A61K 6/69; A61K 6/70

(57) ABSTRACT

A fully dissolvable anticarious remineralizing dental patch for children and a preparation method are provided. The dental patch has a three-layer composite structure: a dissolvable backing protective layer; an anticarious remineralizing active layer; and a pH-responsive layer. All components of the dental patch are food-grade or pharmaceutical excipient-grade raw materials, and the patch is fully dissolvable within 10-30 minutes in the oral environment and requires no manual removal after use. The active layer includes a ternary synergistic system of fluoride-nano-hydroxyapatite-xylitol, which inhibits demineralization, promotes remineralization and inhibits cariogenic bacteria. The active layer is also added with pH-responsive food colorant, which indicates the end point of use intuitively through color change. Formulas are designed for children aged 3-6 years and 6-12 years respectively, and the dental patch is cut along the preset line to adapt to different dental arch sizes.

### CROSS-REFERENCE TO RELATED APPLICATIONS

This application claims priority to Chinese Patent Application No. 202610701376.8, filed on May 20, 2026, which is hereby incorporated by reference in its entirety.

### TECHNICAL FIELD

The present disclosure relates to the technical field of oral care products, and in particular to a fully dissolvable anticarious remineralizing dental patch for children, covering the formula composition, multilayer structure design, preparation process and use method.

### BACKGROUND

Dental caries is one of the most prevalent oral diseases affecting children worldwide. Childhood caries is irreversible. Once a carious cavity is formed, it may progress to pulpitis or even periapical periodontitis, which severely compromises masticatory function, nutrient absorption, eruption of permanent teeth and maxillofacial development.

The high prevalence of childhood caries is attributed to excessive sugar intake, poor self-brushing skills, inadequate oral cleaning and low mineralization degree of dental enamel. More importantly, children have distinct behavioral characteristics from adults. They are unable to comply with prolonged and standardized oral care regimens and face elevated safety risks from accidental swallowing of toothpaste or mouthwash.

At present, commercially available anti-caries products for children are mainly fluoridated toothpaste, remineralizing gels and fluoride varnish preparations.

Fluoridated toothpaste with a fluoride content of 500-1100 ppm prevents caries by means of fluoride ions to boost remineralization and inhibit acid production by cariogenic bacteria. Nevertheless, the efficacy largely depends on children's brushing manner and duration. In practice, children usually brush teeth in an incorrect way and for insufficient time, making it hard to achieve full coverage on tooth surfaces. In addition, infants under three years old are at high ingestion risk, so the usage amount must be strictly controlled. Moreover, immediate rinsing after brushing greatly shortens the residence time of active ingredients on teeth.

Remineralizing gels and professional fluoride varnish preparations, such as CPP-ACP, nHAp and fluoride varnish, require operation by dentists or assistance from guardians. These products suffer from rapid dilution by saliva, short retention on tooth surfaces and fast decline in the concentration of remineralizing ions. Professional fluoridation is normally performed once every 3-6 months, featuring inconvenient operation and poor patient compliance.

In the field of dissolvable oral films, patents of Bsolve Limited (e.g., WO2025/253144A1, CN115768394A) disclose dissolvable oral films containing whitening agents or hydroxyapatite. These products are designed for tooth whitening and dentin desensitization for adults, mainly adopting technologies based on high-concentration peroxides. They are not developed to meet children's demands for anti-caries products, including low ingestion risk, dedicated remineralization systems and optimized flavor.

In terms of remineralizing dental patches, Chinese Patent CN120788920A discloses a dental patch containing regenerated silicon, which is fitted with a non-dissolvable backing layer that needs manual removal, so is not suitable for children. Chinese Patent CN119454532A relates to a multi-layer dental patch added with peroxide whitening agents, and all layers of the patch are non-dissolvable, which is fundamentally different from fully dissolvable products.

In the field of edible films and film-forming materials, expired patents have already disclosed dissolvable films prepared with cellulose film-forming materials. CN104799145A relates to chewable refreshing tablets, rather than patches for attachment on tooth surfaces. CN118236291A applies remineralizing compositions merely as additives in toothpaste, mouthwash and other products, which does not involve independently formed dissolvable film patches, nor is the formulation tailored for children.

The prior art mainly has the following deficiencies.
(1) There are no fully dissolvable anticarious remineralizing dental patches specially developed for children's oral care scenarios. Existing dissolvable oral films are designed for tooth whitening and dentin desensitization in adults. Current remineralizing dental patches contain non-dissolvable backing layers. The available edible films fail to incorporate the synergistic anti-caries system consisting of fluoride, hydroxyapatite and xylitol.
(2) Technical solutions that integrate anti-caries and remineralizing functions into a single dissolvable film system are lacking. Conventional products have single functions and cannot form a complete anti-caries mechanism ranging from demineralization inhibition to remineralization promotion.
(3) Current anti-caries products for children are not equipped with an effective usage feedback mechanism; the efficacy is imperceptible to children and guardians, resulting in poor usage compliance.
(4) Conventional product forms fail to match children's behavioral characteristics. Children tend to resist products requiring manual removal and prolonged static retention on teeth. Therefore, novel products with controllable adhesion duration, no post-use removal and favorable taste are urgently needed.

In addition, existing anti-caries products for children such as fluoridated toothpaste and remineralizing gels rely on tooth brushing or professional operation, and the active ingredients only remain on tooth surfaces for a short time, leading to poor compliance. The existing dissolvable dental patches are not specially optimized for children in terms of low ingestion safety threshold, dental arch size and flavor acceptability. Traditional remineralizing dental patches are fitted with non-dissolvable backing layers that require manual removal, which makes them unsuitable for children. Moreover, there is a lack of products that combine anti-caries and remineralizing functions as well as an intuitive usage feedback mechanism.

### SUMMARY

To address the deficiencies of the prior art, the present disclosure provides a child-specific dental patch integrating full dissolvability, zero swallowing risk, ternary synergistic anti-caries and remineralization, color-change indication and favorable flavor into one single product.

The present disclosure is realized through the following technical solutions.

The fully dissolvable anticarious remineralizing dental patch for children of the present disclosure adopts a three-layer composite structure. All layers are integrated into an integral film via layered coating and drying. All components are food-grade or pharmaceutical excipient-grade materials. The patch is completely dissolved in the oral cavity within 10 to 30 minutes and requires no manual removal after use. The three-layer structure is detailed as follows: the first layer is a dissolvable backing protective layer. This layer includes a fast-dissolving food-grade polymer system, preferably a composite film of pullulan and polyethylene glycol 400. As a natural polysaccharide produced by microbial fermentation, pullulan possesses excellent film-forming property, water solubility and edibility. Polyethylene glycol 400 acts as a plasticizer to endow the film with flexibility and workability. The thickness of the backing layer ranges from 30 to 80 μm, preferably 40 to 60 μm, with a dissolution time of 5 to 10 minutes. The main functions are as follows: protecting the active layer from moisture during storage, providing structural support for easy application and adhesion during use, and gradually dissolving under the action of saliva until fully dissolved.

The second layer is the anti-caries and remineralizing active layer, which serves as the core functional layer of the dental patch. Based on the dry film mass parts, active layer includes the following components: 0.01-0.2 part of fluoride for inhibiting demineralization and promoting the formation of fluorapatite; 1-5 parts of nano-hydroxyapatite to act as a remineralization template and mineral reserve; 10-30 parts of xylitol for inhibiting cariogenic bacteria, synergistically boosting remineralization and imparting sweetness; 40-90 parts of film former to construct the film framework and regulate the dissolution rate; 5-10 parts of plasticizer (such as glycerol or polyethylene glycol) to enhance film flexibility; 1-5 parts of natural flavor agent to deliver fruit flavors acceptable to children; 1-20 parts of water serving as a solvent and removed after drying; and 0.05-0.2 part of food colorant (pH indicator) for indicating the completion of dissolution.

The third layer is a pH-responsive layer. Based on the dry film mass parts,, the pH-responsive layer includes the following components: 0.5-90 parts of chitosan to endow the layer with pH-responsiveness and antibacterial properties; 0.5-20 parts of sodium alginate, which forms a gel network synergistically with chitosan; 1-20 parts of water used as a solvent and removed after drying; and 0.5-10 parts of thickener for adjusting viscosity and film-forming performance.

The dental patch of the present disclosure establishes a ternary synergistic anti-caries and remineralizing system including fluoride, nano-hydroxyapatite and xylitol, which has the following mechanism of action.
(1) Fluoride releases fluoride ions slowly in the oral environment. The ions react with hydroxyapatite in dental enamel by substituting fluoride for hydroxyl groups to form acid-resistant fluorapatite or fluorohydroxyapatite. This reduces the solubility of the enamel surface and inhibits demineralization.
(2) Nano-hydroxyapatite provides preformed calcium phosphate nanocrystals. On the one hand, the nanocrystals act as seed templates for remineralization and directly deposit on the enamel surface to fill micropores caused by demineralization. On the other hand, in the presence of fluoride ions, the nanocrystals induce the formation of more stable fluorohydroxyapatite and improve the acid resistance of the mineralized layer.
(3) As a pentahydroxy alcohol, xylitol cannot be metabolized by oral cariogenic bacteria (primarily Streptococcus mutans) to produce acid. Instead, xylitol interferes with the carbohydrate metabolism of bacteria via competitive inhibition and reduces acid production by dental plaque. Meanwhile, xylitol may form complexes with Ca²⁺in saliva to stabilize calcium ion concentration, and promote the migration and deposition of calcium onto the enamel surface, so as to synergistically enhance the remineralizing effects of fluoride and nHAp. The three components work together to build a complete anti-caries chain covering demineralization inhibition, remineralization promotion and reinforcement of the repaired layer.

In the disclosure, food-grade pH-responsive natural colorant is added into the active layer as a color change indicator. The working principle is as follows: at the initial stage of dental patch attachment, the pH of oral environment is about neutral (6.5-7.5), and the colorant presents the initial color (such as blue-purple). With the release of active ingredients, the pH value of film microenvironment gradually changes from near neutrality to weak acidity, which triggers the color change of colorant (such as blue purple→pink). When the dental patch is completely dissolved, the colorant leaves a very light washable color mark on the tooth surface or tongue surface, indicating the completion of use. Guardians may accurately judge the end point of use without timing; children regard “color change” as gamification feedback, which effectively enhances the use compliance.

The present disclosure adopts the following measures for personalized adaptation and safety design for children.
(1) Age-separated formula design: differentiated fluoride contents and patch sizes are designed for preschool children aged 3-6 years and school-age children aged 6-12 years. The fluoride content of products for 3-6 year-olds is no more than 0.05%, while that for 6-12 year-olds is limited to 0.11%, complying with safety standards for children's toothpaste.
(2) Cuttable personalized adaptation: pre-scored tear lines or dotted score grids are arranged on the edges of the patches. Guardians cut the patches along the preset lines according to children's dental arch length and dentition width for proper fitting.
(3) Complete dissolution and zero residue: all three layers are made of food-grade or pharmaceutical excipient-grade raw materials with oral solubility. No safety risks will occur even if the patch is fully swallowed by children.
(4) Natural flavor system: only naturally derived fruit flavors are used, excluding synthetic essences and irritant menthol. The flavor intensity is kept at a low level acceptable to children, without inducing excessive swallowing.

In terms of sequential release of active ingredients, the three-layer structure of the disclosure realizes sequential release of active ingredients: the backing layer dissolves first within 5-10 minutes to expose and activate the active layer; the active layer continuously releases fluoride, nHAp and xylitol for 10-20 minutes; the pH-responsive layer dissolves last to create an antibacterial environment and buffer local pH value. The sequential dissolution of the three layers greatly extends the residence time of active ingredients on tooth surfaces compared with mouthwash and toothpaste, achieving long-term controlled release.

The fully dissolvable anticarious remineralizing dental patch for children of the present disclosure is prepared by adopting the process of layered film coating, gradient drying and hot-press lamination, including the following steps.

Step 1, preparation and coating of the backing layer: pullulan and polyethylene glycol 400 are dissolved in deionized water according to the formula ratio; the mixture is stirred at 50-60 °C until complete dissolution and then left to stand for defoaming; the film solution is cast onto a polyester release film, and a film applicator is used to control the wet thickness at 40-60 μm; drying is performed to obtain the dry backing layer.

Step 2, preparation and coating of the active layer: the film former is dissolved in deionized water; fluoride solution, nano-hydroxyapatite suspension, xylitol, plasticizer, natural flavor agent and food colorant are added in sequence; the mixture is stirred evenly and then subjected to ultrasonic defoaming; secondary coating is conducted on the dried backing layer; the wet thickness of the active layer is controlled at 80-120 μm, and the gradient drying process is adopted.

Step 3, preparation and coating of the pH-responsive layer: chitosan is dissolved in deionized water, and sodium alginate and thickener are added; the pH value is adjusted to 5.5-6.5; tertiary coating is carried out on the surface of the active layer.

Step 4, hot-press lamination and slitting: the three-layer composite film is placed in a hot press and dried to enhance the bonding force between layers; after cooling, the film is slit into finished dental patches according to preset sizes; each dental patch is individually hermetically sealed and packaged.

Compared with the prior art, the disclosure has the following beneficial effects.
(1) The disclosure resolves the core pain point of poor compliance of existing anti-caries products for children. The fully dissolvable design together with natural fruit flavors eliminates children's resistance; the color change indication system transforms abstract service duration into intuitive visual feedback, and children are more willing to cooperate actively driven by “interesting color change”.
(2) The disclosure constructs a ternary synergistic anti-caries and remineralization system of fluoride-nHAp-xylitol. Fluoride forms an acid-resistant fluorapatite protective layer; nHAp serves as a remineralization template and mineral reserve; xylitol inhibits cariogenic bacteria and synergistically accelerates calcium deposition. The three constituents form a complete anti-caries mechanism from three dimensions: bacteriostasis, demineralization prevention and remineralization promotion.
(3) The safety level of the disclosure satisfies the standard for residence-type oral care products for children. All raw materials adopt food-grade or pharmaceutical excipient grades, and fluoride content receives strict control far below the safety threshold. Total swallowing of the patch brings no safety risk. Age-divided formulation design further safeguards safety for children in different age brackets.
(4) The multi-layer structure realizes the sequential release of active ingredients. Sequential dissolution of the backing layer→active layer→pH-responsive layer extends the residence time of active ingredients on tooth surfaces remarkably relative to mouthwash and toothpaste and achieves long-acting controlled release.
(5) The preparation process features simplicity and adaptability to industrial production. The applied process consists of layered casting, gradient drying and hot-press lamination with mild processing conditions, low energy consumption and moderate equipment requirements, and finished product quality maintains stable controllability.

### BRIEF DESCRIPTION OF THE DRAWINGS

FIG. 1 is a schematic diagram of a three-layer composite structure of a fully dissolvable anticarious remineralizing dental patch for children. In the figure: 1—dissolvable backing protective layer; 2—anti-caries and remineralizing active layer; 3—pH-responsive layer.

FIG. 2 is a comparative diagram of the effect of reducing the enamel white spot area between the dental patch of Embodiment 2 of the present disclosure and the commercially available fluoride toothpaste. In the diagram, the ordinates show the average percentage reduction of enamel white spot area, which is 42.3% in the experimental group (the disclosure) and 18.7% in the control group (the commercially available fluoride toothpaste).

### DESCRIPTION OF EMBODIMENTS

The present disclosure will be described in further detail below in conjunction with embodiments, but the protection scope of the present disclosure is not limited to the following embodiments.

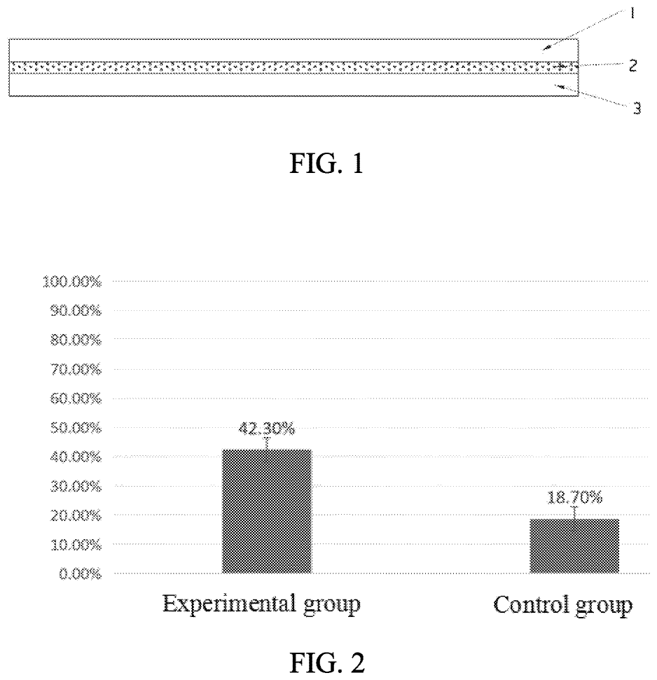

As shown in FIG. 1, a fully dissolvable anticarious remineralizing dental patch for children has a three-layer composite structure, where 1 denotes the dissolvable backing protective layer; 2 denotes the anti-caries and remineralizing active layer; 3 denotes the pH-responsive layer.

### Embodiment 1: Formula for Preschool Children Aged 3-6 Years

In parts by mass of dry film, each layer has the following composition.

Backing layer: 79 parts of pullulan, 15 parts of polyethylene glycol 400, 6 parts of water.

Active layer: 68.88 parts of pullulan (film former), 0.02 part of sodium fluoride, 2 parts of nano-hydroxyapatite, 20 parts of xylitol, 6 parts of glycerol, 2 parts of strawberry flavor, 0.1 part of purple sweet potato anthocyanin (pH indicator), 9 parts of water. pH-responsive layer: 77 parts of chitosan, 15 parts of sodium alginate, 5 parts of water, 3 parts of carbomer.

### Preparation Method

(1) Backing layer: pullulan and polyethylene glycol 400 were dissolved in deionized water. The mixture was stirred at 55 °C until complete dissolution and then left to stand for defoaming. The solution was cast onto a polyester release film with a film thickness of 45 μm set by a film applicator, and then drying treatment was performed.
(2) Active layer: pullulan was dissolved in deionized water. Sodium fluoride solution, nHAp suspension, xylitol, glycerol, strawberry flavor and purple sweet potato anthocyanin were added in sequence. The mixture was stirred evenly and then subjected to ultrasonic defoaming. Secondary coating was carried out on the dry backing layer, followed by drying treatment.
(3) pH-responsive layer: chitosan was dissolved in deionized water, and sodium alginate and carbomer were added. The pH value was adjusted to 6.0. Tertiary coating was implemented on the surface of the active layer, and drying treatment was performed.
(4) Hot-press lamination: the three-layer composite film was dried and then cooled. The film was slit into dental patches and each patch was individually hermetically sealed and packaged.

The dental patch prepared in this embodiment completely dissolved in the oral cavity in about 20 minutes, and the fluoride intake of the single patch was about 0.02 mg, and the flavor was strawberry, which was suitable for children aged 3-6 years.

### Embodiment 2: Formula for School-Age Children Aged 6-12

In parts by mass of dry film, each layer included the following components:

Backing layer: 75 parts of pullulan, 15 parts of polyethylene glycol 400 and 10 parts of water.

Active layer: 58.8 parts of hydroxypropyl methylcellulose (film former), 0.05 part of sodium fluoride, 3 parts of nano-hydroxyapatite, 25 parts of xylitol, 6 parts of glycerol, 2 parts of grape essence, 0.15 part of butterfly bean flower extract (pH indicator) and 5 parts of water.

pH-responsive layer: 82 parts of chitosan, 10 parts of sodium alginate, 5 parts of water and 3 parts of xanthan gum.

### Preparation Method

The preparation process parameters were basically the same as those in Embodiment 1, but the difference was that the wet thickness of the active layer was 90 μm, and the size of the finished dental patch was 1.2 cm×8.0 cm.

The dental patch prepared in this embodiment completely dissolved in the oral cavity in about 25 minutes, and the fluoride intake of the single patch was about 0.04 mg, with a grape flavor, which was suitable for children aged 6-12 years.

### Embodiment 3: Fluorine-Free Alternative Formulation

In the active layer, casein phosphopeptide-amorphous calcium phosphate (CPP-ACP) was used to replace sodium fluoride, and the addition amount was 5 parts. The other components were the same as in Embodiment 1, but the film former (pullulan) was reduced by 3 parts to keep the total parts basically unchanged. The detailed composition is listed below.

Active layer: 65.88 parts of pullulan (film former), 5 parts of CPP-ACP, 2 parts of nano-hydroxyapatite, 20 parts of xylitol, 6 parts of glycerol, 2 parts of strawberry flavor, 0.1 part of purple sweet potato anthocyanin (pH indicator) and 9 parts of water.

The backing layer and the pH-responsive layer were the same as in Embodiment 1.

The preparation process was the same as in Embodiment 1, in which CPP-ACP was added in the last step of active layer preparation at a temperature below 60 °C and stirred evenly.

The dental patch prepared in this embodiment didn't include fluoride, and also had the effect of anti-caries and remineralization, and is suitable for children who are contraindicated by fluoride or prefer fluoride-free products.

### Embodiment 4: Use Effect Test

Thirty children aged 6-12 with early enamel leukoplakia were randomly divided into the experimental group (using the dental patch of Embodiment 2 once a day for 15 minutes) and the control group (brushing teeth with commercially available fluoride toothpaste twice a day). After 4 weeks, the remineralization effect was detected by laser fluorescence caries detector. The results showed that the white spot area in the experimental group decreased by 42.3% on average, and that in the control group decreased by 18.7% on average, and the difference between the two groups was statistically significant (p<0.05) (as shown in FIG. 2). No gum irritation, allergy or other adverse reactions were found during use.

The above is only the optional embodiments of the present disclosure, which does not limit the patent scope of the present disclosure. Any equivalent structure or equivalent process transformation made by using the specification and contents of the present disclosure, or directly or indirectly applied to other related technical fields, are equally included in the patent protection scope of the present disclosure.

What is claimed is:

1. A fully dissolvable anticarious remineralizing dental patch for children, wherein the dental patch has a three-layer composite structure, comprising:
a dissolvable backing protective layer;
an anticarious remineralizing active layer; and
a pH-responsive layer;
wherein all components of the dental patch are food-grade or pharmaceutical excipient-grade raw materials, and the patch is fully dissolvable within 10-30 minutes in the oral environment and requires no manual removal after use.

2. The dental patch according to claim 1, wherein the backing protective layer comprises pullulan and polyethylene glycol 400, with a thickness of 30-80 μm and a dissolution time of 5-10 minutes.

3. The dental patch according to claim 1, wherein in parts by mass of dry film, the active layer comprises 0.01-0.2 part of fluoride, 1-5 parts of nano-hydroxyapatite, 10-30 parts of xylitol, 40-90 parts of film former, 5-10 parts of plasticizer, 1-5 parts of natural flavor agent, 1-20 parts of water and 0.05-0.2 part of food colorant.

4. The dental patch according to claim 3, wherein the food colorant is a pH-responsive natural colorant used to indicate completion of dissolution of the dental patch.

5. The dental patch according to claim 1, wherein in parts by mass of dry film, the pH-responsive layer comprises 0.5-90 parts of chitosan, 0.5-20 parts of sodium alginate, 1-20 parts of water and 0.5-10 parts of thickener.

6. The dental patch according to claim 1, wherein the dental patch is divided into 3-6-year-old preschool children's formula and 6-12-year-old school-age children's formula, wherein a fluoride content of the 3-6-year-old preschool children's formula does not exceed 0.05%, and a fluoride content of the 6-12-year-old school-age children's formula does not exceed 0.11%.

7. The dental patch according to claim 1, wherein an edge of the dental patch is provided with a tear line or dotted score grids used for tailoring according to sizes of children's dental arches.

8. A preparation method for the dental patch according to claim 1, comprising following steps:
step 1, preparing a backing layer coating solution, casting on a release film, and drying to obtain a backing layer dry film;
step 2, coating active layer coating solution on the backing layer dry film, and drying by adopting a gradient drying process;
step 3, coating a surface of the active layer with a pH-responsive layer coating solution, and drying; and
step 4, thermally laminating the three layers of films, slitting, and individually sealing and packaging.

9. The method according to claim 8, wherein a wet thickness of a coated active layer is 80-120 μm.

10. The dental patch according to claim 1, wherein the dental patch is attached to a tooth surface when in use, and a dissolution endpoint is indicated by color change, and the patch dissolves completely after use without removal.
